(19) United States
(12) Patent Application Publication
(10) Pub. No.: US 2019/0318660 A1
(43) Pub. Date: Oct. 17, 2019

(54) OPERATION TRAINING SYSTEM

(71) Applicant: Fanuc Corporation, Yamanashi (JP)

(72) Inventor: Yuuki KIMOTO, Yamanashi (JP)

(73) Assignee: Fanuc Corporation, Yamanashi (JP)

(21) Appl. No.: 16/362,760

(22) Filed: Mar. 25, 2019

(30) Foreign Application Priority Data
Apr. 13, 2018 (JP) JP2018-077275

(51) Int. Cl. G09B 23/28; A61B 34/10; A61B 34/00

(52) U.S. Cl. CPC G09B 23/285; A61B 2034/102; A61B 34/74; A61B 34/10

(57) ABSTRACT

An operation training system includes, a first display device for a first user, a second display device for a second user who learns robot operation from a first user, and a control system configured to move, based on an input made by a second user using a second user input device, a robot displayed on the second display device. The control system is configured to move, based on an input made by the first user using a first user input device, a robot displayed on the first display device. The control system is also configured to display, on the second display device, a motion of the robot based on the input made by the first user using the first user input device.

### CROSS-REFERENCE TO RELATED APPLICATIONS

This application is based on and claims priority to Japanese Patent Application No. 2018-077275 filed on Apr. 13, 2018, the content of which is incorporated herein by reference in its entirety.

### TECHNICAL FIELD

The present invention relates to an operation training system.

### BACKGROUND ART

Conventionally, there is known a training image data communicating device in which image data of roads and buildings that can be seen when a vehicle travels is transmitted from a server device to a client terminal, and the image data is displayed on a display device of the client terminal (cf. PTL 1). In the training image data communicating device, the roads and buildings displayed in the display device of the client terminal move based on an input through an input device of the client terminal.

Further, the image data of the moving roads and buildings in this manner is transmitted to the server device, and the same image is displayed on a display device of the server device. Then, an instructor watching the display device of the server device gives a spoken instruction to a trainee operating the client terminal, and thus training of the trainee is carried out.

There is also known a training device in which a working environment and an operator model are set on a server device, and the working environment on the server device viewed from the operator model is displayed on the display device of the client terminal (cf. PTL 2). As the image displayed on the display device of the client terminal is also displayed on the display device of the server device, the instructor watching the display device of the server device may share an experience of the trainee watching the display device of the client terminal.

### CITATION LIST

### Patent Literature

### {PTL 1}

Japanese Unexamined Patent Application, Publication No. 2002-49299

### {PTL 2}

Japanese Unexamined Patent Application, Publication No. 2002-366021

### SUMMARY OF INVENTION

An operation training system according to one aspect of the present disclosure includes: a first display device for a first user; one or more second display devices each for a second user who learns robot operation from the first user; and a control system configured to move, based on an input made by the second user using a second user input device, a robot displayed on corresponding one of the second display devices, wherein the control system is configured to move, based on an input made by the first user using a first user input device, one of the robot and a robot for the first user displayed on the first display device, and the control system is configured to display, on the second display device, a motion of the one of the robot and the robot for the first user based on the input made by the first user using the first user input device.

### BRIEF DESCRIPTION OF DRAWING

{FIG. 1}

FIG. 1 is a schematic diagram of an operation training system according to one embodiment of the present invention.

{FIG. 2}

FIG. 2 shows an example of display on a second display device of the operation training system according to this embodiment.

{FIG. 3}

FIG. 3 shows an example of display on the second display device of the operation training system according to this embodiment.

{FIG. 4}

FIG. 4 shows an example of display on a first display device of the operation training system according to this embodiment.

{FIG. 5}

FIG. 5 is a block diagram of a first computer of the operation training system according to this embodiment.

{FIG. 6}

FIG. 6 is a block diagram of a second computer of the operation training system according to this embodiment.

{FIG. 7}

FIG. 7 shows an example of display on an operation panel of the operation training system according to this embodiment.

{FIG. 8}

FIG. 8 shows an example of display on the operation panel of the operation training system according to this embodiment.

### DESCRIPTION OF EMBODIMENTS

Hereinafter, an operation training system according to one embodiment of the present invention will be described with reference to the drawings.

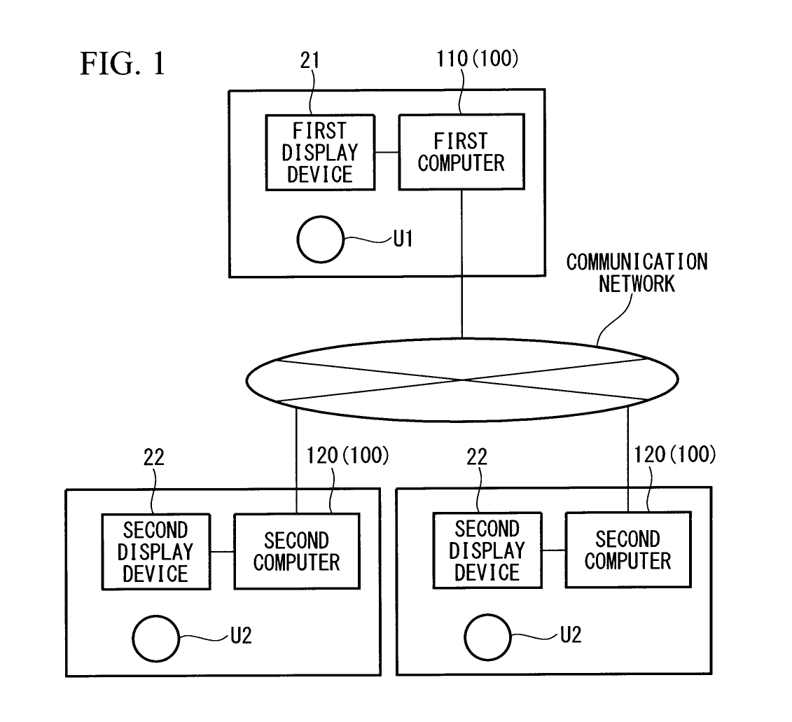

The operation training system according to the present invention includes, as illustrated in FIG. 1, a first display device 21 at which a first user U1 such as a trainer looks, second display devices 22 at which a plurality of second users U2 who learn operation of a robot from the first user U1 respectively look, and a control system 100. The second display devices 22 are placed in spaces different from a space for the first display device 21. For example, as illustrated in FIG. 1, the plurality of second display devices 22 are placed at various places such as houses and working places of the second users U2, at which places the first display device 21 is not placed. The plurality of second users U2 may also respectively look at the plurality of second display devices 22 placed in the same room.

In this embodiment, as illustrated in FIG. 1, the control system 100 includes a first computer 110 connected to the first display device 21, and a plurality of second computers 120 respectively connected to the plurality of second display devices 22.

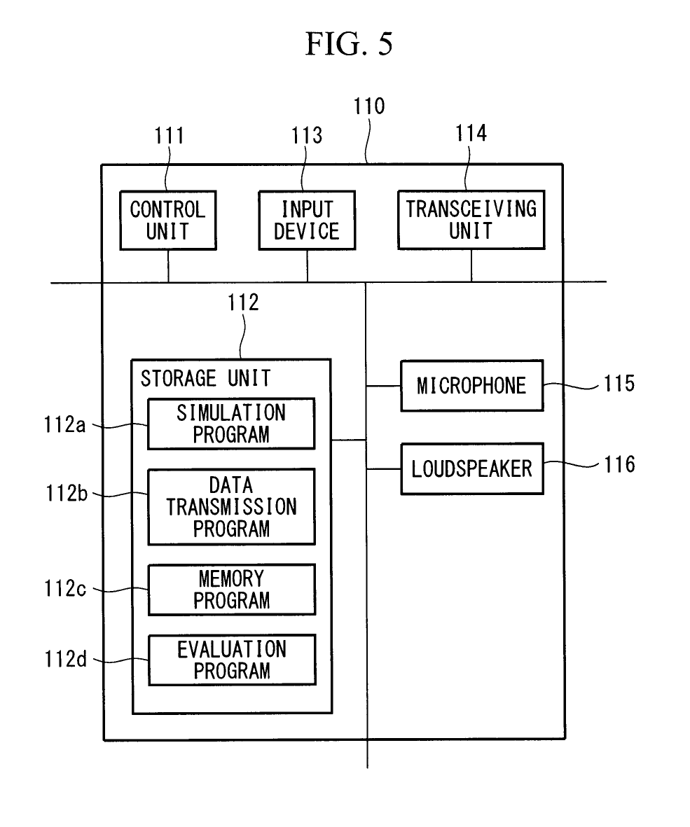

As illustrated in FIG. 5, the first computer 110 includes a control unit 111 having a processor or the like, a storage unit 112 having a non-volatile storage, a ROM, a RAM, or the like, an input device (first user input device) 113 having a keyboard, a mouse, or the like, a transceiving unit 114 configured to transmit and receive data, and having a connecting port to which a communication cable is connected, an antenna, and the like, a microphone 115 as an audio input device, and a loudspeaker 116. The input device 113 may include a touch screen. The touch screen, for example, includes a transparent resistance film provided over a surface of the first display device 21 if the touch screen is of a resistance film type, and includes a transparent electrode film provided over the surface of the first display device 21 if the touch screen is of a capacitive type.

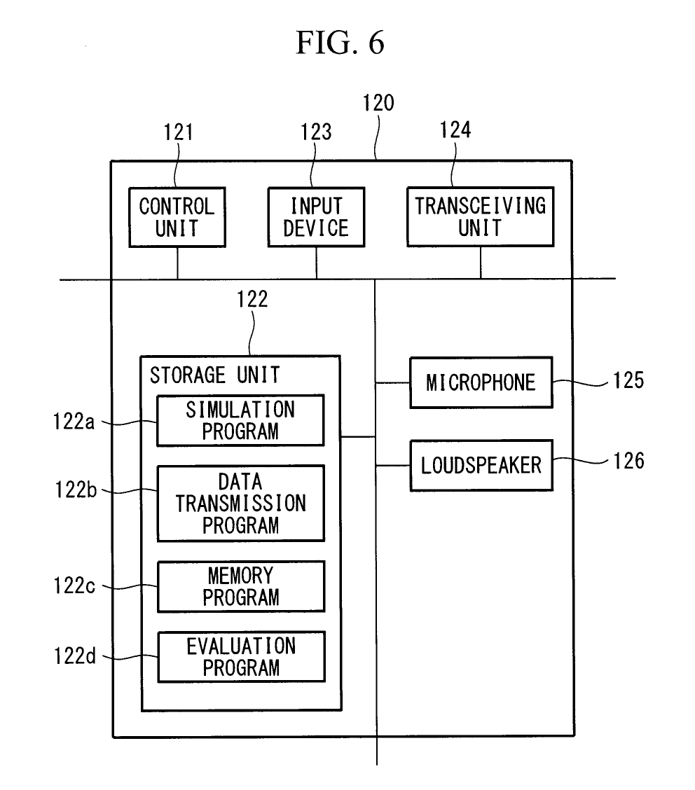

As illustrated in FIG. 6, each of the second computers 120 includes a control unit 121 having a processor or the like, a storage unit 122 having a non-volatile storage, a ROM, a RAM, or the like, an input device (second user input device) 123 having a keyboard, a mouse, or the like, a transceiving unit 124 configured to transmit and receive data, and having a connecting port to which a communication cable is connected, an antenna, and the like, a microphone 125 as an audio input device, and a loudspeaker 126. The input device 123 may include a touch screen similar to that provided for the first computer 110, in place of a keyboard, a mouse, or the like.

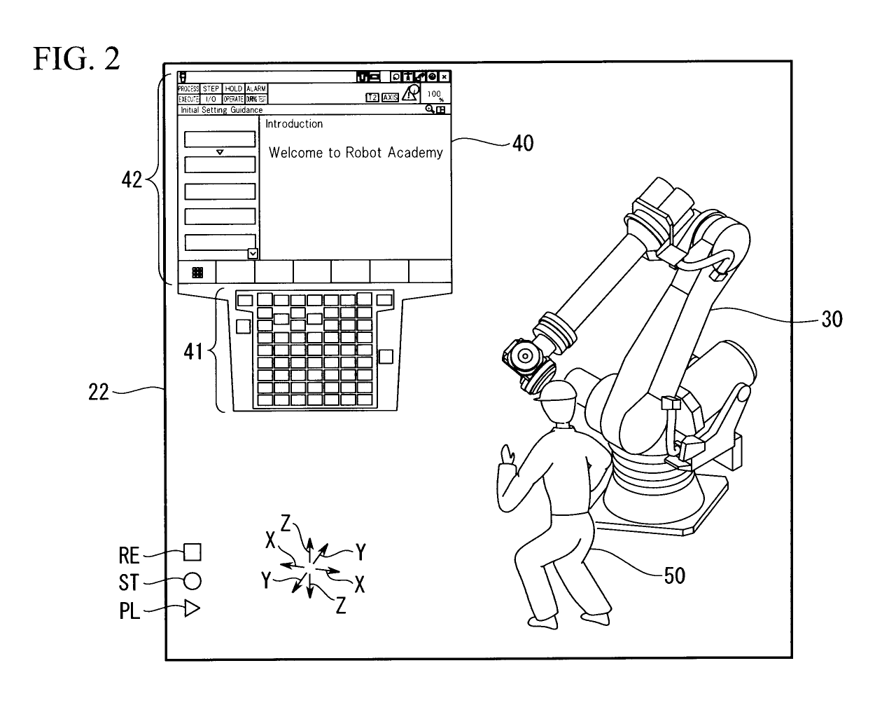

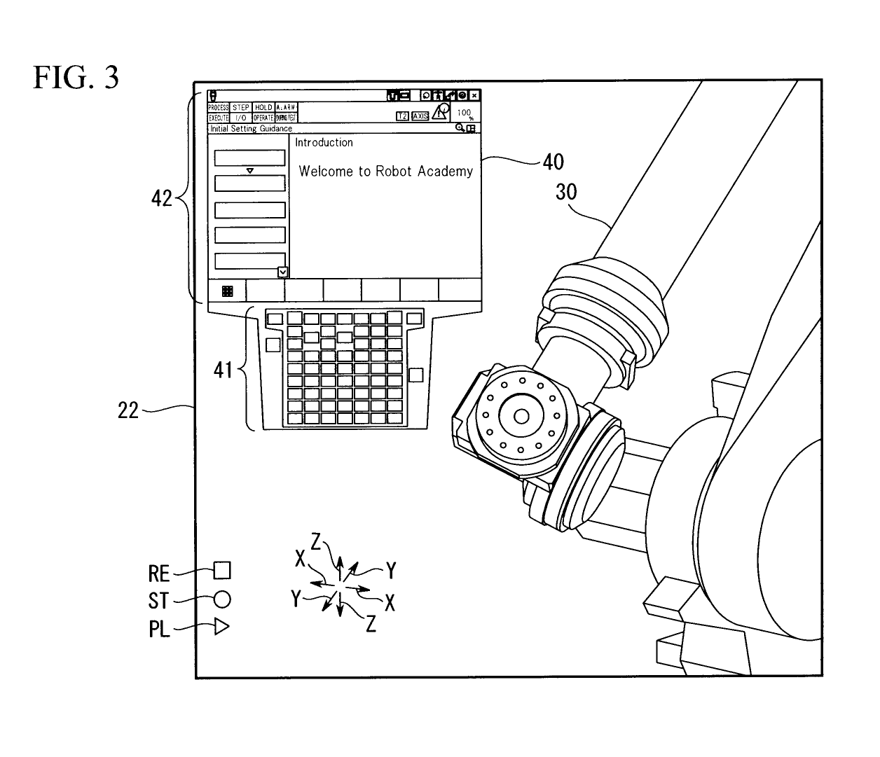

The storage unit 122 of the second computer 120 stores a simulation program 122a for the robot, as well as a robot model, an operation panel model, and a user model. As illustrated in FIG. 2, the control unit 121 displays a robot 30, an operation panel 40, and a user 50 in the corresponding second display device 22, based on the simulation program 122a, the robot model, the operation panel model, and the user model. Here, each of the second users U2 is able to switch perspective of an image displayed on the corresponding second display device 22 by operating the input device 123. In the example shown in FIG. 2, the view point is set at a position where the user 50 and the robot 30 as a whole can be seen. In the example shown in FIG. 3, the view point is set at a position of the eyes of the user 50.

In this embodiment, the robot 30 is an articulated robot. Further, the operation panel 40 includes a first operation section 41 on which a plurality of operation buttons are provided, and a second operation section 42 that displays menu buttons, information relating to a state of the robot 30, and the like. In other words, the operation panel 40 is an actual model of the operation panel including a first operation section having a plurality of operation buttons, and a second operation section of a touch-screen type.

The storage unit 112 of the first computer 110 stores a simulation program 112a for the robot. The simulation program 112a is compatible with the simulation program 122a. As one example, the simulation program 112a and the simulation program 122a are the same program. The storage unit 112 of the first computer 110 stores the robot models, the operation panel models, and the user models that are stored in the plurality of second computers 120. Data for these models may be received from the second computers 120, or may be previously stored in the storage unit 112 of the first computer 110.

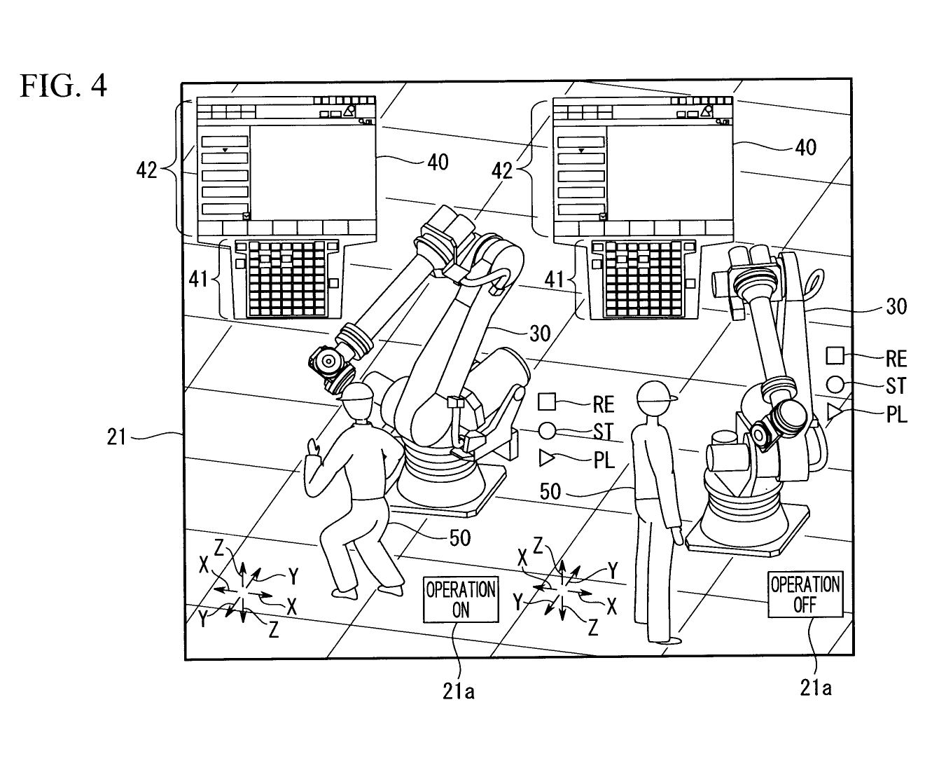

The control unit 111 displays the plurality of robots 30, the plurality of operation panels 40, and the plurality of users 50 on the first display device 21, as illustrated in FIG. 4, based on the simulation program 112a, the robot models, the operation panel models, and the user models. Preferably, the robots 30, the operation panels 40, and the users 50 displayed on the first display device 21 are the same as the robots 30, the operation panels 40, and the users 50 that are displayed on the plurality of second display devices 22. In the example shown in FIG. 4, the plurality of robots 30 are placed side by side, the operation panels 40 are placed in the neighborhood of the respective robots 30, and the user 50 are positioned in the neighborhood of the respective robots 30.

Each of the second users U2 operates the operation panel 40 displayed on the second display device 22 using the input device 123 (robot operation for learning). For example, the second user U2 moves a mouse to position a pointer shown on the second display device 22 over an operation button, a menu button, or the like, and presses a button of the mouse to operate the operation button, the menu button, or the like.

The control unit 121 moves the robot 30 and the operation panel 40 displayed on the second display device 22, based on the simulation program 122a, and based on the operation to the operation panel 40. The movement of the operation panel 40 is to change display of the second operation section 42 and the like.

Further, the second user U2 operates, by using the input device 123, to change a position of a model 50 with respect to the robot 30 displayed on the second display device 22 (change of a user position in learning operation). For example, a pointer is positioned over an arrow X, an arrow Y, and an arrow Z displayed at left bottom on the second display device 22, and then the button of the mouse is pressed.

The control unit 121 moves a position of a face (eye) or a body of the user 50 on the simulation according to the operation, based on the simulation program 122a. If the displayed area of the second display device 22 is adjusted to the field of vision of the user 50 as illustrated in FIG. 3, the control unit 121 may move the position of the face of the user 50 in a front-back direction according to operation of a scroll wheel of the mouse.

The storage unit 122 of the second computers 120 stores an operational data transmission program 122b, and the control unit 121 sequentially transmits operational data (content of operation) for the operation panel 40, motion data of the robot 30, and positional data of the model 50 to the first computer 110 based on the operational data transmission program 122b.

Based on the simulation program 112a, the control unit 111 of the first computer 110 moves the robot 30, the operation panel 40, and the user 50 on the first display device 21 using the corresponding the operational data, the motion data, and the positional data that are sequentially received, and displays an operational situation of an operation buttons and/or a menu button on the operation panel 40. As the display of the operational situation, for example, an operation button, a menu button, or the like that is pressed down by the second user U2 is highlighted. Examples of the highlighting include changing of hue and density of color of these buttons.

With this, the first user U1 as a trainer looks at the first display device 21, and thereby accurately understands robot operation by the second user U2 as the trainee and the position of the user 50 at this time.

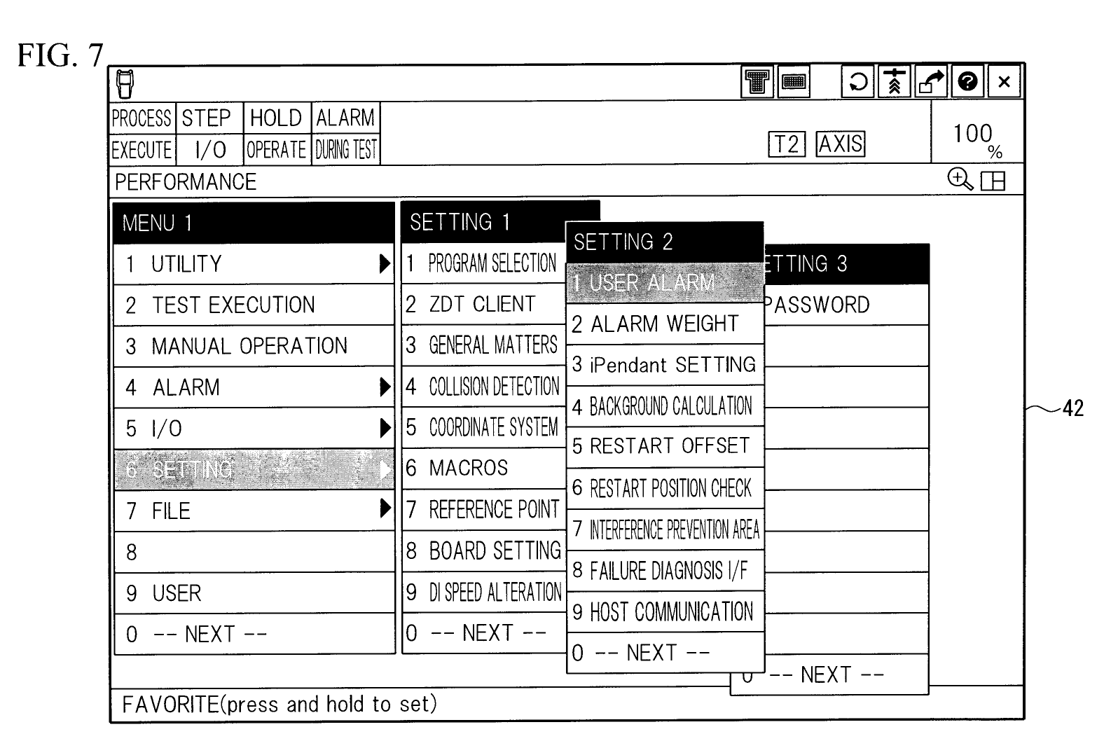

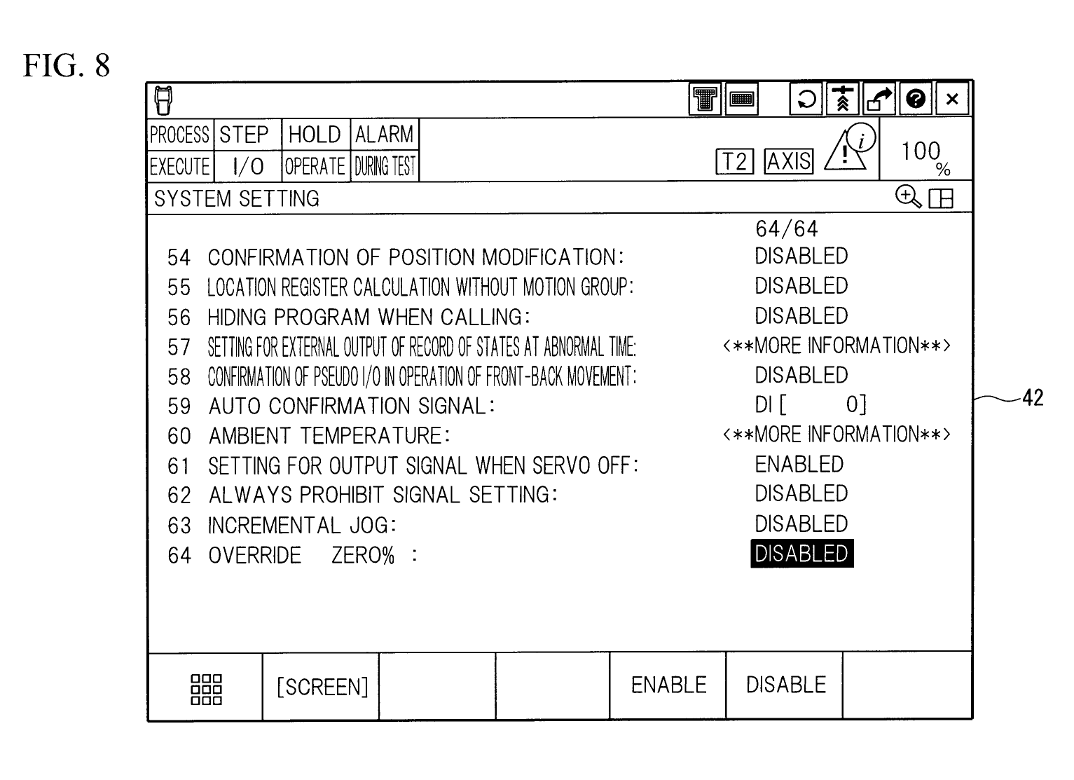

Here, as illustrated in FIG. 2 and FIG. 3, the first operation section 41 of the operation panel 40 has a large number and variety of operation buttons. Further, as illustrated in FIG. 7 and FIG. 8, there is a large number of types and layers of the menu buttons displayed on the second operation section 42 of the operation panel 40, as well as a large number of items for condition setting.

The numbers of the operation buttons, the menu buttons, and the items for condition setting are large because the robot is typically an articulated robot having 6 joints, which allow the robot to make a complicated motion.

For a person who learns robot operation, it is difficult to learn operational procedures and setting procedures for the operation buttons, the menu buttons, and the items for condition setting according to a desired motion of the robot.

Moreover, the robot performs a complicated motion which is a combination of swinging and turning of the plurality of joints. Therefore, a person who does not operate a robot regularly is not able to predict how the joints move, for example, when a distal end portion of the robot moves from one position to another, or does not have a sense to imagine optimal or near optimal movement of each of the joints that is required to allow the robot to make a certain motion. For example, when the distal end portion of the robot is moved, a person who does not operate the robot regularly may not know whether to move the distal end portion of the robot linearly or to make an axial motion.

A person who operates the robot regularly has a sense to imagine optimal or near optimal movement of each of the joints, based on the person's daily experience. For example, a person who works for a robot manufacturer in a department in which robot is regularly operated conducts business assignments related to operation of robots so that robots may satisfy various clients and applications. Therefore, such a person would be able to acquire a sense for robot operation suitable for a variety of situations.

On the other hand, for example, in a factory that employs robots, an operation program for a robot is created when the robot is purchased, or when a new production line is established, which is major timing at which complicated operation of the robot is performed. Further, such operation of the robot is often performed by an engineer of a robot manufacturer. Moreover, types of products treated in the factory are limited, and a situation in which the robot is used is also limited. Here, the operation program is a group of control commands that allow the robot to perform a series of motions.

Therefore, an employee working in the factory using robots rarely acquire a sense for robot operation suitable for a variety of situations.

Further, in a case of vertically articulated robots and horizontally articulated robots, a force applied to the distal end portion of the robot is sequentially transmitted to the joints in a cantilever manner. Therefore, a desirable posture of the robot differs according to weight of a tool attached to the distal end portion of the robot, and weight of an object supported by the robot. A relation between the weight and the posture of the robot influences durability of components of the robot. In addition, it is necessary to consider durability of cables attached to the robot.

Further, each of the joints has its movable range. Therefore, when the distal end portion of the robot sequentially moves to a plurality of positions in various postures, swinging positions and turning positions of the respective joints at each of the positions must be suited for swinging and turning of the respective joints at subsequent positions. In other words, a situation in which the robot is not able to move to the next position as the next position is outside the movable range of each of the joints must be avoided.

In addition, in order to operate the robot safely, knowledge relating to operation that could result in a dangerous motion of the robot is necessary. For example, when an operation program for holding an object by a hand attached to the distal end portion of the robot is to be set, the operator performs the setting while the operator is visually checking positional relation between the hand and the object. Therefore, it is not desirable that the operator lacks the aforementioned knowledge.

Specifically, while an employee working in the factory using robots might have knowledge of operation buttons, menu buttons, and items for condition setting of a control panel 40 to some extent, and is able to move a robot, such an employee in most cases does not have a sense relating to setting of an operation program, and/or sufficient experience and knowledge for safe manual operation of the robot when the operator is near the robot. Further, as such an employee performs complicated robot operation only occasionally, the employee forgets a major part of knowledge, experience, and the like that have been obtained in complicated robot operation by the time of the next robot operation.

The first user U1 as a trainer has, for example, a sense for robot operation suited for various situations, as well as sufficient experience and knowledge relating to operation that could result in a dangerous motion of the robot. The first user U1 is able to operate the operation panel 40 displayed on the first display device 21 using the input device 113 (robot operation for instruction). By the operation, the operation panel 40 and the robot 30 displayed on the first display device 21 move. At this time, it is possible to move the user 50 displayed on the first display device 21 (change of the user position during teaching operation). It should be noted that, in this embodiment, the first display device 21 displays operation ON/OFF buttons 21a respectively correspond to the plurality of robots 30, and the robot 30 for which the operation ON/OFF button 21a is turned ON becomes operable.

The first user U1 moves a mouse to position a pointer shown on the first display device 21 over an operation button, a menu button, or the like on the operation panel 40, and presses a button of the mouse to operate the operation button, the menu button, or the like.

The control unit 111 moves the robot 30 displayed on the first display device 21, based on the simulation program 112a, and based on the operation to the operation panel 40 displayed on the first display device 21.

Further, the first user U1 is able to change the position of the user 50, similarly to the case of the second user U2. For example, it is possible to change the position of the user 50 with respect to the robot 30, by positioning the pointer over the arrow X, the arrow Y, and the arrow Z displayed near the user 50 on the first display device 21, and then pressing the button of the mouse.

The storage unit 112 of the first computer 110 stores an operational data transmission program 112b, and the control unit 111 sequentially transmits the operational data for the operation panel 40, the motion data of the robot 30, and the positional data of the model 50, to one of the second computers 120 corresponding to the operation panel 40 that has been operated based on the operational data transmission program 112b.

The control unit 121 of the second computer 120 based on the simulation program 122a, moves the robot 30, the operation panel 40, and the user 50 on the second display device 22 using the operational data (content of operation), the motion data, and the positional data that are sequentially received from the first computer 110, and displays the operational situation of the operation buttons and/or the menu button on the operation panel 40. As the display of the operational situation, for example, the pointer operated by the first user U1 is displayed, and an operation button, a menu button, or the like that has been pressed down by the first user U1 is highlighted. Examples of the highlighting include changing of hue and density of color of these buttons.

As described above, in this embodiment, by watching appropriate robot operation by the first user U1 as a trainer on the second display device 22, the second user U2 is able to learn the appropriate robot operation. Further, the second user U2 is able to learn the appropriate robot operation, while staying at a place different from a place where the first user U1 is.

When the robot operation for instruction and the change of the user position during teaching operation are performed, the first user U1 is able to provide voice such as an instruction and explanation using the microphone 115 for the second user U2 to be instructed. According to operation to the input device 113, the first computer 110 is set to a mode for providing voice for all of the plurality of second users U2, or to a mode for providing voice for only selected one or more of the plurality of second users U2.

The loudspeaker 126 of the second computers 120 outputs voice transmitted from the first computer 110. Here, the second user U2 is also able to transmit voice of questions and the like to the first user U1 using the microphone 125.

In this manner, as the first user U1 as a trainer is able to talk with the second user U2, the second user U2 is able to learn the robot operation efficiently.

The first computer 110 may display a text entry field on the first display device 21, and the second computer 120 may display a text entry field on the second display device 22. Further, letters input in the text entry field on the first display device 21 using the input device 113 may be displayed on the second display device 22 by the second computer 120, and letters input in the text entry field on the second display device 22 using the input device 123 may be displayed on the first display device 21 by the first computer 110. In this case, the same effect as in the case in which voice is used may be obtained as well.

Further, when the robot operation for instruction and the change of the user position during teaching operation are performed, the control unit 111 is able to, based on a memory program 112c stored in the storage unit 112, store the robot operation for instruction and the change of the user position during teaching operation according to an input to the input device 113 in the storage unit 112. At this time, the control unit 111 is also able to store voice input to the microphone 115 in the storage unit 112.

For example, when a record button RE on the first display device 21 is operated by positioning the pointer and pressing the button of the mouse, processing for storing the robot operation for instruction and the change of the user position during teaching operation is started. On the other hand, when a stop button ST on the first display device 21 is operated by positioning the pointer and pressing the button of the mouse, the processing for storing is terminated. It is also possible to operate a record button or a stop button of the input device 113.

The control unit 111 of the first computer 110 transmits the stored data of the robot operation for instruction and the stored data of the change of the user position during the teaching operation to the second computers 120, and the control unit 121 of the second computer 120 stores the received data of the robot operation for instruction and the received data of the change of the user position during the teaching operation in the storage unit 122.

On the other hand, the control unit 121 is able to, based on a memory program 122c stored in the storage unit 122, store the robot operation for instruction and the change of the user position during teaching operation according to an input to the input device 123 in the storage unit 122. At this time, the control unit 121 is also able to store voice input to the microphone 115 in the storage unit 122.

For example, when a record button RE on the second display device 22 is operated by moving the pointer and pressing the button of the mouse, processing for storing the robot operation for instruction and the change of the user position during teaching operation is started. On the other hand, when a stop button ST on the second display device 22 is operated by moving the pointer and pressing the button of the mouse, the processing for storing is terminated. It is also possible to operate a record button or a stop button of the input device 123.

Then, when a play button PL on the second display device 22 is operated, the robot operation for instruction and the change of the user position during teaching operation are reproduced on the second display device 22. By operating a play button PL on the first display device 21, reproduction is started on the first display device 21.

The storing of the robot operation for instruction and the change of the user position during teaching operation as described above is advantageous for efficient and correct learning of appropriate robot operation, and leads to reduction of time and effort of the first user U1. It should be noted that a motion of the robot 30 according to the robot operation for instruction may also be stored at the same time. On the other hand, it is possible to store only the robot operation for instruction.

As described above, the control system 100 has a function for storing the content of operation by the first user U1 carried out using the input device 113 to the operation panel 40 displayed on the first display device 21, and a motion of the robot 30 according to the content of operation in the storage units 112 and 122 provided for the control system 100, as well as a function for displaying the content of operation by the first user U1 and the motion of the robot 30 according to the content of operation that have been stored on the second display device 22.

Therefore, the second user U2 is able to repeatedly watch the content of operation by the first user U1 and the motion of the robot 30 according to the content of operation. With this, it is possible to learn the robot operation that is difficult to learn as described above both theoretically and intuitively, which is extremely advantageous in order to acquire a sense for robot operation.

On the other hand, when the robot operation for learning and the change of the user position during learning operation are performed, the control unit 121 is able to, based on the memory program 122c stored in the storage unit 122, store the robot operation for learning and the change of the user position during learning operation according to the input to the input device 123 as described above in the storage unit 122.

The control unit 121 of the second computer 120 transmits the stored data of the robot operation for learning and the stored data of the change of the user position during the learning operation to the first computer 110, and the control unit 111 of the first computer 110 stores the received data of the robot operation for learning and the received data of the change of the user position during the learning operation in the storage unit 112.

On the other hand, the control unit 111 is able to, based on the memory program 112c stored in the storage unit 112, store the robot operation for learning and the change of the user position during learning operation according to an input to the input device 113 as described above in the storage unit 112.

Then, when a play button PL on the second display device 22 is operated, the robot operation for learning and the change of the user position during learning operation are reproduced on the second display device 22. By operating a play button PL on the first display device 21, reproduction is started on the first display device 21.

It should be noted that a menu screen for selecting a robot operation among a plurality of robot operations during the teaching operation or during the learning operation may be displayed.

The storing function of the robot operation for learning and the change of the user position during learning operation as described above is advantageous for efficient and correct learning of appropriate robot operation. The second user U2 is able to observe the own operation objectively, and compare the own operation with the operation by the first user U1. With this, it becomes easier for the second user U2 to learn points to be improved in the own robot operation, and this is advantageous for efficient learning by the second user U2.

Here, there is a case in which the second user U2 creates an operation program including a dangerous motion of the robot, or an operation program with which the robot cannot move beyond the movable range of each of the joints. When the robot 30 moves based on such an operation program, and the robot makes a dangerous motion or reaches a motion limit, the control unit 121 stops the robot 30. This stopping of the robot 30 is displayed on the first display device 21 by the control unit 111. Therefore, the first user U1 is able to know whether or not the robot making the dangerous motion or reaching the motion limit easily.

It should be noted that the control unit 111 may evaluate the robot operation by each of the second users U2, based on an evaluation program 112d stored in the storage unit 112. For example, the control unit 111 performs evaluation about a dangerous motion for the robot operation by the second user U2. In this case, the storage unit 112 stores, for example, a danger degree table in which a plurality of motion patterns of the robot 30, positions of the model 50, and indices regarding a degree of danger are associated. Further, the control unit 111 refers to the danger degree table, and derives a degree of danger of each motion included in the robot operation by the second user U2. In place of the danger degree table, or in addition to the danger degree table, it is possible to use a formula for evaluating a degree of danger in order to calculate a degree of danger. The danger degree table or the formula for evaluating a degree of danger is one example of an evaluation reference.

Further, the control unit 121 performs evaluation about an operational sense of the robot operation by the second user U2. In this case, the storage unit 122 stores, for example, an operational sense table in which a plural types of the robot operation are associated with a plurality of motion patterns of the robot. The plural types of the robot operation in the operational sense table are respectively assigned with evaluation points. Further, the control unit 121 refers to the operational sense table, and derives an operational sense of each motion included in the robot operation by the second user U2. In place of the operational sense table, or in addition to the operational sense table, it is possible to use a formula for evaluating an operational sense in order to calculate an operational sense. The operational sense table or the formula for evaluating an operational sense is one example of the evaluation reference.

One example of the operational sense relates to the durability of components, cords, and the like of the robot. In this case, the plural types of the robot operation in the operational sense table are respectively assigned with evaluation points relating to the durability of the robot.

Another example of the operational sense relates to the movable ranges of the joints. In this case, the plural types of the robot operation in the operational sense table are respectively assigned with evaluation points relating to the movable ranges of the joints.

Further, the control unit 111 displays, based on the evaluation program 112d stored in the storage unit 112, an evaluation result (evaluated value) on the first display device 21. Often, the first user U1 fails to notice an error in the robot operation to an extent such that the robot 30 does not stop. Employing this configuration allows the first user U1 to teach in a more detailed manner, and the first user U1 is able to easily determine which one of the second users U2 should be taught. This is highly advantageous for efficient learning of appropriate robot operation.

Employing this configuration also allows the second user U2 to efficiently learn appropriate robot operation, even when there are a large number of the second users U2 that the first user U1 is to instruct. In particular, at a stage in which a large number of the second users U2 learn basic operations, it is possible to employ a machine instructor as the first user U1, and to divide the plurality of second users U2 depending on the evaluation.

Further, the control unit 111 may display evaluation results respectively for evaluation subjects on the first display device 21 and/or the second display devices 22. Examples of the evaluation subjects include evaluation relating to a degree of danger of a type 1 robot, evaluation relating to operability of the type 1 robot, evaluation relating to durability of the type 1 robot, evaluation relating to movable ranges of the type 1 robot, and evaluation relating to a degree of danger of a type 2 robot. In this case, the control unit 111 has evaluation references for the respective evaluation subjects.

It should be noted that the storage unit 122 of the second computer 120 may store a corresponding evaluation program 122d, and the control unit 121 may perform the same processing as the control unit 111 of the first computer 110 regarding the evaluation.

It should also be noted that the control unit 121 may be configured to store, based on the memory program 122c stored in the storage unit 122, a motion of the robot 30 including a dangerous motion, a position of the user 50 at this time, and operation to the operation panel 40 relating the motion and the position in the storage unit 122. In this case, the second user U2 is able to confirm a dangerous point in own robot operation by watching the motion and such of the stored motion of the robot 30, and this is highly advantageous in order to improve safety of the robot operation.

Further, a plurality of examples of the dangerous motion may be stored in the storage unit 112 or the storage unit 122. In this case, the second user U2 is able to confirm a dangerous point in own robot operation by watching motions of the robot 30 stored as the dangerous motion, positions of the user 50 at these times, and the like, and this is highly advantageous in order to improve safety of the robot operation.

Moreover, a head-mounted display may be connected to the second computer 120. In this case, the control unit 121 displays an image viewed from the model 50 on the head-mounted display. Preferably, this image is a three-dimensional image. In particular, the control unit 121 displays a motion of the robot 30 including the dangerous motion described above in the head-mounted display. With tis, the second user U2 is able to experience a dangerous situation close to an actual situation, and this is highly advantageous for developing a sense of the second user U2 relating to dangerous operation.

Here, in another example, the operation training system includes one first display device 21 and one second display device 22. In this case, when the second user U2 as a skilled robot operator creates an operation program for a new product, the second user U2 is able to learn robot operation one on one from the first user U1 who has a sense of the robot operation suitable for various situations. Similarly, in this case, the effects described above based on the memory programs 112c and 122c are advantageous. Further, using the evaluation programs 112d and 122d, the first user U1 is able to evaluate and understand the skill of the second user U2 in a more detailed manner, and this is highly advantageous for efficient learning of appropriate robot operation.

It should be noted that the control unit 111 of the first computer 110 may display a robot for the first user different from the robot 30 operated by the second user U2 on the first display device 21, and the first user U1 may operate the robot for the first user. Then, the control unit 111 may sequentially transmit motion data of the robot for the first user to the second computer 120, and the control unit 121 of the second computer 120 may move the robot for the first user or the robot 30 based on the received motion data. In this case, the effects described above may also be obtained.

It should be noted that the control system 100 may include a server device. In this case, the server device includes a part or all of the functions of the first computer 110 and the second computer 120. Further, the control system 100 may include only the first computer 110. In this case, the first computer 110 includes all of the functions of the second computer 120.

From the above-described embodiments, the following aspects of the present disclosure are derived.

An operation training system according to one aspect of the present disclosure includes: a first display device for a first user; one or more second display devices each for a second user who learns robot operation from the first user; and a control system configured to move, based on an input made by the second user using a second user input device, a robot displayed on corresponding one of the second display devices, wherein the control system is configured to move, based on an input made by the first user using a first user input device, one of the robot and a robot for the first user displayed on the first display device, and the control system is configured to display, on the second display device, a motion of the one of the robot and the robot for the first user based on the input made by the first user using the first user input device.

According to this aspect, the motion of the robot moved based on the input by the first user such as a trainer is displayed on the second display device. Therefore, by watching appropriate robot operation by the first user on the second display device, the second user such as a trainee is able to learn the appropriate robot operation. Here, an articulated robot makes a motion that is difficult to predict, and an operation method of the robot in order to optimize tasks such as work efficiency, safety, durability of components, cords, and the like of the robot, may vary depending on conditions such as the type of the robot and the content of operation of the robot. Therefore, it is not easy to improve the skill of the second user by communicating solutions and points of these tasks by voice alone.

By contrast, according to this aspect, the second user may understand differences between a motion of the robot operated by the second user and a motion of the robot operated by the first user through an image displayed on the second display device. With this, such differences and the robot operation by the first user remains as images in the memory of the second user, and thus the second user is able to learn appropriate robot operation efficiently.

In the aspect described above, preferably, a content of operation is displayed on the second display device, the content of operation being performed by the first user using the first user input device to an operation panel displayed on the first display device.

According to this aspect, it is possible for the second user to understand differences between a motion of the robot operated by the second user and a motion of the robot operated by the first user through an image displayed on the second display device, and to understand the differences associated with the operation of the operation panel by the first user. This is highly advantageous for efficient learning of appropriate robot operation.

In the aspect described above, preferably, the control system is configured to display, out of the content of operation, an operational situation of either one or both of an operation button and a menu button of the operation panel displayed on the first display device, and an action of the operation panel, on an operation panel displayed on the second display device.

According to this aspect, it is possible for the second user to understand differences between a motion of the robot operated by the second user and a motion of the robot operated by the first user through an image displayed on the second display device, and to understand the operation of the operation panel by the first user through an image. More specifically, the second user is able to carefully observe operation of the operation panel by the first user, as well as to watch a motion of the robot operated by such operation. This is highly advantageous for efficient learning of appropriate robot operation.

In the aspect described above, preferably, the control system has functions of: storing, in a storage unit of the control system, a content of operation performed by the first user using the first user input device to an operation panel displayed on the first display device, and a motion of the one of the robot and the robot for the first user according to the content of operation, and displaying, on the second display device, the stored content of operation performed by the first user, and the motion of the one of the robot and the robot for the first user according to the content of operation.

In this case, the second user is able to repeatedly watch the content of operation by the first user and the motion of the robot according to the content of operation. With this, it is possible to learn the robot operation that is difficult to learn as described above both theoretically and intuitively, which is extremely advantageous in order to acquire a sense for robot operation.

In the aspect described above, preferably, the control system has functions of: storing, in a storage unit of the control system, a content of operation performed by the second user using the second user input device to an operation panel displayed on the second display device, and a motion of the robot according to the content of operation, and displaying, on the second display device, the stored content of operation performed by the second user, and the motion of the robot according to the content of operation.

In this case, the second user is able to repeatedly and objectively watch the content of own operation and the motion of the robot according to the content of operation. According to this aspect, the second user is able to compare the own operation with the operation by the first user, and it is possible for the second user to efficiently learn appropriate robot operation through the comparison.

In the aspect described above, preferably, the control system is configured to evaluate robot operation by the second user based on a predetermined evaluation reference, and to display a result of the evaluation on the first display device.

Often, the first user fails to notice an error by the second user in the robot operation to an extent such that the robot does not stop. Especially when there are a plurality of second users, this aspect allows the first user to easily determine which one of the second users should be instructed. This is highly advantageous for efficient learning of appropriate robot operation.

In the aspect described above, preferably, the control system is configured to display a motion of the robot including a dangerous motion on the second display device.

According to this aspect, the second user is able to understand the motion of the robot including a dangerous motion through an image, and this is advantageous in order to acquire a sense for safe robot operation.

In the aspect described above, preferably, the control system is configured to display a motion of the robot including a dangerous motion on a head-mounted display worn by the second user.

According to this aspect, the second user is able to experience the motion of the robot including a dangerous motion close to an actual situation. A motion of the robot and atmosphere immediately before entering a dangerous stage vary depending on types of the robot, operation carried out by the robot, and the like. Experiencing the motion of the robot including a dangerous motion close to an actual situation is highly advantageous in order to acquire a sense for safe robot operation, and desirably to learn how to avoid such a situation.

In the aspect described above, preferably, the control system is configured to move a user model displayed near the robot on the second display device based on the input to the second user input device, and the control system is configured to display the user model near the robot on the first display device, the user model moves based on the input to the second user input device.

According to this aspect, the second user is able to perform robot operation while considering own position near the robot, and the first user is able to confirm the position of the second user during robot operation. This is advantageous in order to learn safe operation of the robot.

According to the aforementioned aspects, it is possible to learn an operation method suitable for ensuring safety in robot operation as well as for situations.

### REFERENCE SIGNS LIST

U1 First user
U2 Second user
21 First display device
22 Second display device
30 Robot
40 Operation panel
50 User
100 Control system
110 First computer
111 Control unit
112 Storage unit
112a Simulation program
112b Data transmission program
112c Memory program
112d Evaluation program
113 Input device (first user input device)
120 Second computer
121 Control unit
122 Storage unit
122a Simulation program
122b Data transmission program
122c Memory program
122d Evaluation program
123 Input device (first user input device)

1. An operation training system comprising:
a first display device for a first user;
one or more second display devices each for a second user who learns robot operation from the first user; and
a control system configured to move, based on an input made by the second user using a second user input device, a robot displayed on corresponding one of the second display devices, wherein
the control system is configured to move, based on an input made by the first user using a first user input device, one of the robot and a robot for the first user displayed on the first display device, and
the control system is configured to display, on the second display device, a motion of the one of the robot and the robot for the first user based on the input made by the first user using the first user input device.

2. The operation training system according to claim 1, wherein
the control system is configured to display a content of operation on the second display device, the content of operation being performed by the first user using the first user input device to an operation panel displayed on the first display device.

3. The operation training system according to claim 2, wherein
the control system is configured to display, out of the content of operation, an operational situation of either one or both of an operation button and a menu button of the operation panel displayed on the first display device, and an action of the operation panel, on an operation panel displayed on the second display device.

4. The operation training system according to claim 1, wherein
the control system has functions of:
storing, in a storage unit of the control system, a content of operation performed by the first user using the first user input device to an operation panel displayed on the first display device, and a motion of the one of the robot and the robot for the first user according to the content of operation, and
displaying, on the second display device, the stored content of operation performed by the first user, and the motion of the one of the robot and the robot for the first user according to the content of operation.

5. The operation training system according to claim 1, wherein
the control system has functions of:
storing, in a storage unit of the control system, a content of operation performed by the second user using the second user input device to an operation panel displayed on the second display device, and a motion of the robot according to the content of operation, and
displaying, on the second display device, the stored content of operation performed by the second user, and the motion of the robot according to the content of operation.

6. The operation training system according to claim 1, wherein
the control system is configured to evaluate robot operation by the second user based on an evaluation reference, and to display a result of the evaluation on the first display device.

7. The operation training system according to claim 1, wherein
the control system is configured to display a motion of the robot including a dangerous motion on the second display device.

8. The operation training system according to claim 1, wherein
robot including a dangerous motion on a head-mounted display worn by the second user.

9. The operation training system according to claim 1, wherein
the control system is configured to move a user model displayed near the robot on the second display device based on the input to the second user input device, and
the control system is configured to display the user model near the robot on the first display device, the user model moves based on the input to the second user input device.
